(19) United States
(12) Patent Application Publication
(10) Pub. No.: US 2017/0015973 A1
(43) Pub. Date: Jan. 19, 2017

(54) COLLAGEN HYDROGEL KIT AND METHODS OF MAKING A COLLAGEN HYDROGEL

(71) Applicant: MedTrain Technologies, LLC, Burlington, NC (US)

(72) Inventor: Albert J. Banes, Hillsborough, NC (US)

(21) Appl. No.: 15/124,137

(22) PCT Filed: Mar. 9, 2015
(86) PCT No.: PCT/US15/19462

§ 371 Date: Sep. 7, 2016

Related U.S. Application Data
(60) Provisional application No. 61/950,540, filed on Mar. 10, 2014.

(51) Int. Cl. C12N 5/00

(52) U.S. Cl. CPC C12N 5/0068; C12N 2533/54

(57) ABSTRACT

Provided is a kit and method for preparing a collagen hydrogel media matrix. The kit includes a collagen, a cell growth medium, a serum supplement, an organic chemical buffering agent, and an aqueous base. The method includes the steps of preparing a first mixture comprising a cell growth medium, a serum supplement, and an organic buffering agent, preparing a second mixture comprising a collagen and an aqueous base, testing the pH of the second mixture by adding additional aqueous base and combining the first and second mixtures.

### CROSS-REFERENCE TO RELATED APPLICATIONS

This application claims priority to U.S. Provisional Patent Application No. 61/950,540, filed Mar. 10, 2014, the contents of which are incorporated herein in reference in their entirety.

### BACKGROUND OF THE INVENTION

Field of the Invention

The present invention relates to collagen hydrogels. More specifically, the present invention relates to a kit and method for making a collagen hydrogel.

Description of Related Art

Collagen hydrogels are in broad use in today's clinical, tissue engineering and cell biology fields as scaffolds, labware coating, cell encapsulation materials, and injectables. Examples of such collagen hydrogels and methods for preparing them are disclosed in U.S. Patent Application Publication No. 2013/0149782 to Banes, which is hereby incorporated by reference in its entirety.

However, scientists and lab workers routinely encounter considerable difficulty in preparing the hydrogels. Notably, the inventor has recognized that it is common for even experienced cell culturists to require months of practice before they become successful in casting cell-populated hydrogels for use in tissue engineering. Moreover, even if the gel material sets, the cells are commonly found to be not viable due to inadequate mixing of the components and pH gradients in the gel, which may drive the cells to apoptosis and death.

As the need for highly purified Type I and Type II collagens grows in not only the basic science market, but in the medical injectables market, there will be a need for a reliable kit adapted for good manufacturing practices to make collagen hydrogels of a defined composition for use in basic science and medical uses as a carrier for injectable drugs that require persistence in a given region of the body.

### SUMMARY OF THE INVENTION

In order to address the aforementioned issues, provided is a kit and method for preparing a collagen hydrogel medium matrix. The present invention may be implemented in producing a Collagel® hydrogel (which includes atelopeptide containing collagen in combination with other kit components described herein below) or a ThermaCol® hydrogel (which includes a mixture comprising approximately 10% telopeptide containing collagen and approximately 90% atelopeptide containing collagen), however, this feature is not intended to be limiting, and it is envisioned that the present invention may be used in all manners of collagen hydrogels. Surprisingly, the inventors have found that a collagen hydrogel may be reliably made by using premeasured sodium hydroxide solution at a concentration of approximately 0.1 M. It is an objective of the present invention to allow even beginners at preparing collagen hydrogels to achieve greater success rates than are typically achieved by more experienced scientists mixing their own components. Particularly, the inventor has recognized that experienced researchers generally utilize a more concentrated base, such as aqueous 1 M NaOH, and that this practice often results in variable gelation due to inadequate distribution of the base throughout the gel and local toxic effects of the concentrated base.

Moreover, in embodiments of the present kit and method that utilize a mixture of telopeptide-containing collagen as well as atelo-peptide-containing collagen in a specific ratio, such as a mixture of approximately 90% atelopeptide-containing collagen and 10% telopeptide-containing collagen, a controllable gelation kinetic may be provided, and much longer collagen fibrils can be achieved. The end result of the kit formulation is that the researcher is able to reliably cast collagen hydrogels with included cells, that gel reproducibly, may maintain cell viability over a period of weeks, and have reproducible compaction kinetics so that a tissue engineered construct may be implanted in a host animal if desired.

The kit may comprise the following components: a collagen, a cell growth medium, a serum supplement, an organic chemical buffering agent, and an aqueous base. Preferably, the base is a strong base at a concentration of approximately 0.05 to 0.15 M. In such aspects, the base may be selected from a group including, but not limited to, NaOH, KOH, LiOH, ZnOH, Ca(OH)_{2,} or any combination thereof.

Alternatively, a weak base may be substituted for the strong base and the concentration of the weak base should be an appropriate concentration to titrate the acid to a pH of 7.2 to 7.6, with the ideal pH being 7.4. Potential weak bases include, but are not limited to, NaHCO₃, TRIS base, alanine, glycine, ammonia, dimethylamine, ethylamine, or any combination thereof.

The base can be NaOH at a concentration of approximately 0.1 M. The preferred cell growth medium is a Minimum Essential Medium (“MEM”) such as 5X MEM. The aqueous base solution can also further comprise a cell growth medium, such as 5X MEM. The serum supplement may be selected from a group including but not limited to fetal bovine serum, newborn bovine serum, adult bovine serum, fetal porcine serum, fetal equine serum, fetal carpine serum, fetal rabbit serum, fetal ovine serum, fetal chicken serum, or any combination thereof, with the preferred serum supplement being fetal bovine serum.

The organic buffering agent can be aqueous HEPES at a concentration of 10-30 mM, however, other buffering agents may be substituted such as TAPSO, TES, or MOPS. The buffering agent can have a pKa between 7.0 and 8.0 at 25° C.

The collagen can be an atelopeptide-containing type I collagen at a concentration ranging from 1.0 to 4.4 mg/mL in 0.006 to 0.020 M aqueous HCl.

Each of the components of the present kit, including the collagen, the cell growth medium, the serum supplement, the organic chemical buffering agent, and the aqueous base is preferably provided in a separate, sealed container. The components of the kit are ideally provided together in a single package, and the package should be stored at approximately 4° C.

The components of the kit can be provided in the following ratios: 6.0 to 10.0 parts of the collagen, in solution, 1.0 to 3.0 parts of the cell growth medium, 0.5 to 1.5 parts of the serum supplement, 0.1 to 0.4 parts of the organic buffering agent, and 0.2 to 0.8 parts of the aqueous base.

In another aspect of the present invention, the kit may further comprise a telopeptide-containing type I collagen. The telopeptide-containing type I collagen is provided at a concentration of 2.0 to 4.0 mg/mL in 0.004 to 0.016 M aqueous HCl.

When telopeptide-containing type I collagen is included in the kit, the components of the kit are can be provided in the following ratios: 6.0 to 10.0 parts collagen solution, 1.0 to 3.0 parts of the cell growth medium, 0.5 to 1.5 parts of the serum supplement, 0.1 to 0.4 parts of the organic buffering agent, 0.2 to 0.8 parts of the aqueous solution comprising the base, and 1.5 to 2.5 parts of the telopeptide-containing type I collagen, in solution.

In another aspect of the present invention, a method is provided for preparing a collagen hydrogel media matrix. The method may comprise the following steps: preparing a first mixture comprising a cell growth medium, a serum supplement, and an organic buffering agent, preparing a second mixture comprising a collagen and an aqueous base, testing the pH of the second mixture, adjusting the pH of the second mixture to a range of 6.8 to 7.6 by adding additional aqueous base in increments of approximately 1.0 to 4.0 μL, and combining the first and second mixtures.

It should be noted that cells may be added to the mixture after mixing of the initial components and the mixture is brought to the correct pH by addition of the base. In such circumstances, the volume of water added to the mixture initially should be reduced to account for the additional volume of the cells in a small volume of MEM.

The base can be a strong base at a concentration ranging from 0.05 to 0.25 M, the cell growth medium is a 5X MEM, the serum supplement is a fetal bovine serum or bovine serum from newborn animals or adults, the organic chemical buffering agent is an aqueous HEPES at a concentration of 10 to 30 mM, and the collagen is an atelopeptide-containing type I collagen at a concentration ranging from 2.0 to 4.0 mg/mL in 0.004 to 0.020 M aqueous HCl. The pH of the second mixture can be adjusted by adding aqueous base in 2.5 μL increments, and the aqueous base solution can further comprise a cell growth medium.

The first mixture may comprise 130 to 200 parts of the MEM, 50 to 110 parts of the serum supplement, and 10 to 30 parts of the organic buffering agent, and the second mixture may comprise 60 to 84 parts of the aqueous base and 650 to 750 parts of the collagen at a concentration ranging from 2.0 to 4.0 mg/mL in 0.004 to 0.016 M aqueous HCl. The aqueous base can be a strong base at a concentration ranging from 0.05 to 0.25 M, however, a weak base may be substituted for the strong base. In such aspects, the concentration of the weak base can be an appropriate concentration to titrate the acid to a pH of 7.2 to 7.6, with the ideal pH being 7.4.

In another aspect of the present method, the second mixture may further comprise a telopeptide-containing type I collagen. The collagen can be provided at a concentration ranging from 2.0 to 4.0 mg/mL in 0.004 to 0.016 M aqueous HCl, and the telopeptide-containing type I collagen is preferably provided at a concentration ranging from 2.0 to 4.0 mg/mL in 0.004 to 0.016 M aqueous HCl.

In this aspect of the invention, the first mixture can comprise 130 to 200 parts of the MEM, 70 to 90 parts of the serum supplement, and 10 to 30 parts of the organic buffering agent, and the second mixture may comprise, 60 to 84 parts of the aqueous base, 600 to 660 parts of the collagen, at a concentration ranging from 2.0 to 4.0 mg/mL in 0.004 to 0.016 M aqueous HCl, and 40 to 100 parts of the telopeptide-containing type I collagen at a concentration ranging from 2.0 to 4.0 mg/mL in 0.004 to 0.016 M aqueous HCl. The aqueous base can be a strong base at a concentration ranging from 0.05 to 0.25 M, but, a weak base may be substituted as described above.

In another aspect, the kit may comprise, instead of a serum supplement, a viscous solution, matching the oncotic pressure, measured in osmolarity, equivalent to that of the serum component. Non-limiting oncotically equivalent solutions to serum include bovine serum albumin, hydroxyethyl starch, dextran, and gelatin, or any combination thereof.

Aspects of the invention will now be described in the following numbered clauses:

Clause 1: A kit for preparing a collagen hydrogel medium matrix comprising the following components: a collagen; a cell growth medium; a serum supplement; an organic chemical buffering agent; and an aqueous base.

Clause 2: The kit of clause 1, wherein the base is a strong base at a concentration of approximately 0.05 to 0.25 M.

Clause 3, The kit of clauses 1 or 2, wherein the base is selected from the group consisting of NaOH, KOH, LiOH, ZnOH, Ca(OH)₂, or any combination thereof.

Clause 4: The kit of clause 1, wherein the base is selected from the group consisting of NaHCO₃, TRIS base, alanine, glycine, ammonia, dimethylamine, ethylamine, or any combination thereof.

Clause 5: The kit of any of clauses 1-3, wherein the base is NaOH at a concentration of approximately 0.1 M.

Clause 6: The kit of any of clauses 1-5, wherein the serum supplement is selected from the group consisting of fetal bovine serum, newborn bovine serum, adult bovine serum, fetal porcine serum, fetal equine serum, fetal carpine serum, fetal rabbit serum, fetal ovine serum, fetal chicken serum, or any combination thereof.

Clause 7: The kit of any of clauses 1-5, wherein the aqueous base further comprises a cell growth medium.

Clause 8: The kit of any of clauses 1-7, wherein the cell growth medium is a minimum essential medium (“MEM”).

Clause 9: The kit of any of clauses 1-3 and 5-8, wherein the base is NaOH, the cell growth medium is 5X Minimal Essential Medium (“MEM”), the serum supplement is fetal bovine serum or bovine serum from newborn animals or adults, the organic chemical buffering agent is aqueous HEPES at a concentration of approximately 10-30 mM, and the collagen is atelopeptide-containing type I collagen at a concentration ranging from 1.0 to 4.4 mg/mL in 0.006 to 0.020 M aqueous HCl.

Clause 10: The kit of any of clauses 1-9, wherein each of the collagen, the cell growth medium, the serum supplement, the organic chemical buffering agent, and the aqueous base is provided in a separate, sealed container.

Clause 11: The kit of any of clauses 1-10, wherein the components are provided in the following ratios: 6.0 to 10.0 parts of the collagen, in solution; 1.0 to 3.0 parts of the cell growth medium; 0.5 to 1.5 parts of the serum supplement; 0.1 to 0.4 parts of the organic buffering agent; and 0.2 to 0.8 parts of the aqueous base.

Clause 12: The kit of any of clauses 1-11, further comprising a telopeptide-containing type I collagen.

Clause 13: The kit of clause 12, wherein the telopeptide-containing type I collagen is provided at a concentration of 2.0 to 4.0 mg/mL in 0.004 to 0.016 M aqueous HCl.

Clause 14: The kit of clauses 12 or 13, wherein the components are provided in the following ratios: 6.0 to 10.0 parts collagen solution; 1.0 to 3.0 parts cell growth medium; 0.5 to 1.5 parts serum supplement; 0.1 to 0.4 parts organic buffering agent; 0.2 to 0.8 parts aqueous solution comprising the strong base; and 1.5 to 2.5 parts telopeptide-containing type I collagen, in solution.

Clause 15: The kit of any of clauses 1-14 wherein the aqueous base is 0.1 M NaOH provided at 4 volume percent of the total volume of solutions in the kit.

Clause 16: The kit of any of clauses 1-10 and 12 wherein the components are provided in the following amounts: 16 volume percent of the cell growth medium, 8 volume percent of the serum supplement, 2 volume percent of the organic buffering agent, 70 volume percent of the collagen and 4 volume percent of the aqueous base.

Clause 17: A method of preparing a collagen medium matrix comprising: preparing a first mixture comprising a cell growth medium, a serum supplement, and an organic buffering agent; preparing a second mixture comprising a collagen and an aqueous base; testing the pH of the second mixture; adjusting the pH of the second mixture to a range of 6.8 to 7.6 by adding additional aqueous base in increments of approximately 1.0 to 4.0 μL, and combining the first and second mixtures.

Clause 18: The method of clause 17, wherein the base is a strong base at a concentration ranging from 0.05 to 0.25 M, the cell growth medium is 5X MEM, the serum supplement is fetal bovine serum or bovine serum from newborn animals or adults, the organic chemical buffering agent is aqueous HEPES at a concentration of 10 to 30 mM, and the collagen is atelopeptide-containing type I collagen at a concentration ranging from 2.0 to 4.0 mg/mL in 0.004 to 0.020 M aqueous HCl.

Clause 19: The method of clauses 17 or 18, wherein the pH of the second mixture is adjusted by adding aqueous base in 2.5 μL increments.

Clause 20: The method of any of clauses 17-19, wherein the aqueous base further comprises a cell growth medium.

Clause 21: The method of any of clauses 17-20, wherein: the first mixture comprises 130 to 200 parts of the MEM, 50 to 110 parts of the serum supplement, and 10 to 30 parts of the organic buffering agent; the second mixture comprises, 60 to 84 parts of the aqueous base and 650 to 750 parts of the collagen at a concentration ranging from 2.0 to 4.0 mg/mL in 0.004 to 0.016 M aqueous HCl; and wherein the aqueous base is a strong base at a concentration ranging from 0.05 to 0.25 M.

Clause 22: The method of any of clauses 17-21, wherein the second mixture further comprises a telopeptide-containing type I collagen.

Clause 23: The method of clause 22, wherein the collagen is provided at a concentration ranging from 2.0 to 4.0 mg/mL in 0.004 to 0.016 M aqueous HCl, and the telopeptide-containing type I collagen is provided at a concentration ranging from 2.0 to 4.0 mg/mL in 0.004 to 0.016 M aqueous HCl.

Clause 24: The method of clause 22, wherein the first mixture comprises 130 to 200 parts of the MEM, 70 to 90 parts of the serum supplement, and 10 to 30 parts of the organic buffering agent; the second mixture comprises, 60 to 84 parts of the aqueous base, 600 to 660 parts of the collagen, at a concentration ranging from 2.0 to 4.0 mg/mL in 0.004 to 0.016 M aqueous HCl, and 40 to 100 parts of the telopeptide-containing type I collagen at a concentration ranging from 2.0 to 4.0 mg/mL in 0.004 to 0.016 M aqueous HCl; and wherein the aqueous base is a strong base at a concentration ranging from 0.05 to 0.25 M.

Clause 25: A kit for preparing a collagen hydrogel medium matrix comprising the following components: a collagen; a cell growth medium; an oncotically equivalent solution to serum; an organic chemical buffering agent; a cell growth medium; and 0.1 M NaOH.

Clause 26: The kit of clause 25, wherein the oncotically equivalent solution is selected from the group consisting of bovine serum albumin, hydroxyethyl starch, dextran, and gelatin, or any combination thereof.

Clause 27: The kit of clauses 25 or 26, wherein the cell growth medium is a minimum essential medium (“MEM”).

Clause 28: The kit of any of clauses 25-27, wherein the cell growth medium is 5X Minimal Essential Medium (“MEM”), the oncotically equivalent solution is bovine serum albumin, the organic chemical buffering agent is aqueous HEPES at a concentration of approximately 10-30 mM, and the collagen is atelopeptide-containing type I collagen at a concentration ranging from 1.0 to 4.4 mg/mL in 0.006 to 0.020 M aqueous HCl.

Clause 29: The kit of any of clauses 25-28, wherein each of the collagen, the cell growth medium, the oncotically equivalent solution to serum, the organic chemical buffering agent, and the 0.1 M NaOH is provided in a separate, sealed container.

Clause 30: The kit of any of clauses 25-29, wherein the components are provided in the following ratios: 6.0 to 10.0 parts of the collagen, in solution; 1.0 to 3.0 parts of the cell growth medium; 0.5 to 1.5 parts of the oncotically equivalent solution to serum; 0.1 to 0.4 parts of the organic buffering agent; and 0.2 to 0.8 parts of the 0.1M NaOH.

Clause 31: The kit of any of clauses 25-30, further comprising a telopeptide-containing type I collagen.

Clause 32: The kit of clause 31, wherein the telopeptide-containing type I collagen is provided at a concentration of 2.0 to 4.0 mg/mL in 0.004 to 0.016 M aqueous HCl.

Clause 33: The kit of clauses 31 or 32, wherein the components are provided in the following ratios: 6.0 to 10.0 parts collagen solution; 1.0 to 3.0 parts cell growth medium; 0.5 to 1.5 parts oncotically equivalent solution to serum; 0.1 to 0.4 parts organic buffering agent; 0.2 to 0.8 parts aqueous solution comprising the 0.1M NaOH; and 1.5 to 2.5 parts telopeptide-containing type I collagen, in solution.

Clause 34: The kit of any of clauses 25-33 wherein the 0.1 M NaOH is provided at 4 volume percent of the total volume of solutions in the kit.

Clause 35: The kit of any of clauses 25-29, 31-32, and 34, wherein the components are provided in the following amounts: 16 volume percent of the cell growth medium, 8 volume percent of the oncotically equivalent solution to serum, 2 volume percent of the organic buffering agent, 70 volume percent of the collagen and 4 volume percent of the aqueous base.

### DETAILED DESCRIPTION OF THE INVENTION

The above, and other features and characteristics of the present invention, as well as the methods of operation and functions of the related elements of structures and the combination of parts and economies of manufacture, will become more apparent upon consideration of the following description and the appended claims with reference to the accompanying tables and examples, all of which form a part of this specification. It is to be expressly understood, however, that the examples are for the purpose of illustration and description only and are not intended as a definition of the limits of the invention. As used in the specification and the claims, the singular form of “a”, “an”, and “the” include plural referents unless the context clearly dictates otherwise.

Unless otherwise indicated, all numbers expressing dimensions, physical characteristics, quantities of ingredients, reaction conditions and so forth, used in the specification and claims are to be understood as being modified in all instances by the term “about.” Accordingly, unless indicated to the contrary, the numerical values set forth in the following specification and claims can vary depending upon the desired properties sought to be obtained by the present invention. At the very least, each numerical parameter should at least be construed in light of the number of reported significant digits and by applying ordinary rounding techniques.

Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more and ending with a maximum value of 10 or less, e.g., 5.5 to 10 or 3.2 to 7.8.

In order to better facilitate understanding, the present invention is described below with reference to specific examples. The below-mentioned examples are not intended to be limiting, but rather to be illustrative of preferred configurations of the present invention.

### EXAMPLE 1

In a first example, a kit and a method are disclosed for preparing a standard collagen hydrogel medium matrix. In this non-limiting example, the kit is provided as a package with each reagent being in a separate, sealed container in one of three volumes provided in Table 1. In this non-limiting example, the kit is provided in three separate sizes, each designed to facilitate the production of a different number of cultures, but the ratio of the various components of the kit is maintained.

The kit and method for preparing a collagen medium matrix may be used in creating bioartificial cell-populated hydrogels (BAT) in linear tissue train culture plates.

TABLE 1

| | | Volume (mL) | | |
|---|---|---|---|---|
| | | Mini | Midi | Maxi |
| Component | Chemical | Kit | Kit | Kit |
| Collagel ® | Type I Collagen | 8 | 16 | 32 |
| | (3.2 mg/ml in 0.012M HCl) | | | |
| Reagent A | 5X MEM | 2 | 4 | 8 |
| Reagent B | Fetal Bovine Serum | 1 | 2 | 4 |
| Reagent C | 20 mM HEPES | 0.25 | 0.5 | 1 |
| Reagent D | 0.1M NaOH in 5X MEM | 0.5 | 1 | 2 |

A Mini-Kit has approximately enough solution to make BATs in eight 6-well linear Tissue Train ® culture plates, a midi kit has enough for sixteen 6-well plates, and a maxi kit for thirty-two 6-well plates.

Table 2 shows volumes of the reagents that may be used in the preparation of the collagen hydrogel media matrix based on the tray size, with the first column being an example of the recommended ratio of reagents:

TABLE 2

| | | Volume | | | | |
|---|---|---|---|---|---|---|
| | | (μL) | 6-Well Tissue | | 24-Well HTP | |
| | | (Example | Train ® | | Tissue Train ® | |
| Reagent: | Chemical: | Ratios) | (200 μL/BAT) | | (50 μL/BAT) | |
| A | 5X MEM | 160 | 288 | μL | 288 | μL |
| B | Fetal | 80 | 144 | μL | 144 | μL |
| | Bovine | | | | | |
| | Serum | | | | | |
| C | 20 mM | 20 | 36 | μL | 36 | μL |
| | HEPES | | | | | |
| Collagel ® | Type I | 700 | 1260 | μL | 1260 | μL |
| | Collagen | | | | | |
| | (3.2 mg/ml | | | | | |
| | in 0.012M | | | | | |
| | HCl) | | | | | |
| D | 0.1M NaOH | 40 | 72 | μL | 72 | μL |
| | in 5X MEM | | | | | |

| | | | | | |
|---|---|---|---|---|---|
| Total | 1000 | 1800 | μL | 1800 | μL |

It should be noted that all reagents should be kept at approximately 4 degrees C., or the preparation should be made on ice.

In accordance with this non-limiting example, preparation takes place in the following steps, using the example ratio of reagents:
A. In a 1.5 mL tube, combine reagents A, B, and C forming a first mixture.
B. In a 5.0 mL tube add reagent D to the Collagel® and mix well, forming a second mixture (thoroughly mixed solution should become light peach in color).
C. Remove 10 μL, of the second mixture and apply to pH paper to ensure pH is neutral (7.0-7.4 is ideal).
   i. If the pH is not in the desired range, add 2.5 μL Reagent D, check the pH again, and repeat step C(i) until the pH is in desired range.
D. Combine the first and second mixtures and mix well by pipetting.

### EXAMPLE 2

In a second example, a kit and a method are disclosed for preparing a collagen hydrogel medium matrix further comprising telopeptide containing collagen. In this non-limiting example, the kit is provided as a package with each reagent being in a separate, sealed container the volumes provided in Table 3. In this non-limiting example, the kit is provided in three separate sizes, each designed to facilitate the production of a different number of cultures, but the ratio of the various components of the kit is maintained.

The kit and method for preparing a collagen medium matrix may be used in creating bioartificial cell-populated hydrogels (BAT) in linear tissue train culture plates.

TABLE 3

| | | Volume (mL) | | |
|---|---|---|---|---|
| | | Mini | Midi | Maxi |
| Component | Chemical | Kit | Kit | Kit |
| Collagel ® | Atelopeptide-containing | 8 | 16 | 32 |
| | Type I Collagen | | | |
| | (3 mg/mL in 0.01M HCl) | | | |
| ThermaCol ® | Telopeptide-containing Type | 2 | 4 | 8 |
| Additive | I Collagen (3 mg/mL in | | | |
| | 0.01M HCl) | | | |
| Reagent A | 5X MEM | 2 | 4 | 8 |
| Reagent B | Fetal Bovine Serum | 1 | 2 | 4 |
| Reagent C | 20 mM Hepes | 0.25 | 0.5 | 1 |
| Reagent D | 0.1M NaOH in 5X MEM | 0.5 | 1 | 2 |

A Mini- Kit has approximately enough solution to make BATs in eight 6-well linear Tissue Train ® culture plates, a midi kit has enough for sixteen 6-well plates, and a maxi kit for thirty- two 6-well plates.

Table 4 shows volumes of reagents that may be used in the preparation of the collagen hydrogel media matrix based on the tray size, with the first column being an example of the recommended ratio of reagents:

TABLE 4

| | | Volume | | | | |
|---|---|---|---|---|---|---|
| | | (μL) | 6-Well Tissue | | 24-Well HTP | |
| | | (Example | Train ® | | Tissue Train ® | |
| Reagent: | | Ratios) | (200 μL/BAT) | | (50 μL/BAT) | |
| A | 5X MEM | 160 | 288 | μL | 288 | μL |
| B | Fetal Bovine | 80 | 144 | μL | 144 | μL |
| | Serum | | | | | |
| C | 20 mM | 20 | 36 | μL | 36 | μL |
| | HEPES | | | | | |
| Collagel ® | Atelopeptide- | 630 | 1134 | μL | 1134 | μL |
| | containing | | | | | |
| | Type I | | | | | |
| | Collagen | | | | | |
| | (3 mg/mL in | | | | | |
| | 0.01M HCl) | | | | | |
| ThermaCol ® | Telopeptide- | 70 | 126 | μL | 126 | μL |
| Additive | containing | | | | | |
| | Type I | | | | | |
| | Collagen (3 | | | | | |
| | mg/mL in | | | | | |
| | 0.01M HCl) | | | | | |
| D | 0.1M NaOH | 40 | 72 | μL | 72 | μL |
| | in 5X MEM | | | | | |
| Total | | 1000 | 1800 | μL | 1800 | μL |

It should be noted that all reagents should be kept at approximately 4 degrees C., or the preparation should be made on ice.

In accordance with this non-limiting example, preparation takes place in the following steps, using the example ratio of reagents:
A. In a 1.5 mL tube, combine reagents A, B, and C forming a first mixture.
B. In a 5.0 mL tube, combine the Collagel® solution with the ThermaCol® additive, add reagent D, and mix well, forming a second mixture (thoroughly mixed solution should become light peach in color).
C. Remove 10 μL of the second mixture and apply to pH paper to ensure pH is neutral (7.0-7.4 is ideal).
   i. If the pH is not in the desired range, add 2.5 μL Reagent D, check the pH again, and repeat step C(i) until the pH is in desired range.
D. Combine the first and second mixtures and mix well by pipetting.

### EXAMPLE 3

In this Example, various protein solutions (identified below in Table 5) are substituted for the serum (Reagent B in Examples 1 and 2). These solutions are the oncotic equivalents to serums identified above.

TABLE 5

| | | | Stock | In-gel |
|---|---|---|---|---|
| | | | concen- | concen- |
| | | | trations | trations |
| Substance | Source | Details | (mg/mL) | (mg/mL) |
| Hydroxyethyl | Sigma | MW not | 30, 40, 50 | 2.4-4.0 |
| starch | | published | | |
| Dextran | Sigma | ~70,000 MW | 30, 40, 50 | 2.4-4.0 |
| Bovine serum | Fisher | Fraction V | 40, 50 | 2.4-4.8 |
| albumin | | | | |
| Gelatin | Fisher | Type A | 20, 30, 40 | 1.6-3.2 |
| Fetal Bovine | Atlanta | | (see above | (see above |
| Serum | Biologicals | | Examples) | Examples) |

The starch, albumin and other molecules included as substitutes for serum are calculated to approximate the osmolarity of blood, or cell culture medium with 5-10% serum added. This osmolarity is in the range of 320 milliosmoles (mOsm).

The efficacy of each of the above substitutes, or oncotically-equivalent substitutes for serum, may be used in a Collagel® hydrogel. As above the reagents utilized are Collagel®, 0.1M NaOH, 5x MEM, 1M HEPES, and the respective oncotically-equivalent serum substitute. Gels are prepared, for example, using the below protocol:

1 mL of Collagel® is prepared, by combining 700 μL Collagel® and 40 μL 0.1M NaOH, mixed well by pipetting, and pH is checked to ensure proper neutrality. Then 160 μL 5x MEM, 20 μL 1M HEPES, and 80 μL of oncotically-equivalent substitute are combined. The Collagel® mixture is combined with the mixture of the remaining components, and the final solution is mixed well by pipetting, and gelation occurs. The combined solutions (final solution) can be held at 30° C. for up to 30 minutes. Gelation can occur at 40° C. However, with addition of telopeptide-containing Type I Collagen, gels can form more rapidly. As with Example 2, telopeptide-containing Type I collagen can also be added to the Collagel® solution.

Hydrogels formed using the above substitutes for serum provide a suitable scaffold for cell survival and growth. Moreover, kits containing the various oncotic pressure substitutes can be considerably more convenient, as the substitutes can be stored, long-term, at 4° C., which is the temperature at which the other reagents of the kit are stored. This is in contrast to the serum supplement (of Examples 1 and 2), which must be stored long-term at −20° C.

### EXAMPLE 4

Any of the above-described protocols and hydrogels can be used for a cell-populated hydrogel for culture plates, for example Flexcell® Linear Tissue Train® Culture Plates (Flexcell International, Burlington, N.C., USA). A model protocol is described below, though those of skill in the art will appreciate that modifications to the protocol can be undertaken. Such protocols are available, for example, Tech Report 100: Tissue Train® Culture System—A Method for Culture and Mechanical Loading of Cells in a 3D Matrix, by Banes et al. (2015), incorporated herein by reference in its entirety. However, briefly, a potential protocol is described below.

First, cells are trypsinized by aspiration of general growth medium (GM), washing the monolayer once with 1X DPBS, and addition of 2.0 mL 0.05% Trypsin with EDTA followed by incubation at 37° C., 5% CO2 for 5 minutes.

Next, cells are resuspended with an additional 8.0 mL of GM followed by centrifugation to remove trypsin containing medium and a final resuspension in 10 mL GM.

Cells are then counted. A concentration of 1.0×10³ cells/μL of Collagel®-media matrix is typical; however, however those of skill in the art will appreciate that this ratio will change depending on cell type.

Next, the desired amount of cells that will be used for seeding a single culture plate are sedimented for linear bioartificial cell-populated hydrogels (BAT). In a non-limiting example: 1.8×10⁶ total cells may be used for one 6-well plate batch (200 μL/BAT). As above, those of skill in the art will appreciate that alterations in this amount of cells is possible based on conditions and cell type.

Next, the plate may be positioned on a loading station, for example a Trough Loader™ (Flexcell International), and in a Loading Station™. Proper seating should be confirmed to ensure a sealed vacuum.

Next, a bioreactor or incubator can be used to apply vacuum. Suitable bioreactors/incubators include the Flexcell® FX-5000™ Tension System (Flexcell International). While those of skill in the art will appreciate that various parameters may be utilized, in a non-limiting embodiment, the bioreactor/incubator can be set to initiate a maximum elongation (˜90 KPa) static pull down. Vacuum can be applied for any suitable amount of time, so long as gelation has occurred. For example, and without limitation more than 1 hour, more than 2 hours, or more than 3 hours. The vacuum can be released slowly by 2% elongation of maximum every 6 seconds until 0% elongation is achieved, though as above, those of skill in the art will appreciate that these parameters can be modified based on cell type and other conditions.

The invention has been described with reference to the preferred embodiment. Obvious modifications and alterations will occur to others upon reading and understanding the preceding detailed description. It is intended that the invention be construed as including all such modifications and alterations insofar as they come within the scope of the appended claims or the equivalents thereof.

The invention claimed is:

1. A kit for preparing a collagen hydrogel medium matrix comprising the following components:
a collagen;
a cell growth medium;
a serum supplement;
an organic chemical buffering agent; and
an aqueous base.

2. The kit of claim 1, wherein the base is a strong base at a concentration of approximately 0.05 to 0.25 M.

3. The kit of claim 1 or 2, wherein the base is selected from the group consisting of NaOH, KOH, LiOH, ZnOH, Ca(OH)₂, or any combination thereof.

4. The kit of claim 1, wherein the base is selected from the group consisting of NaHCO₃, TRIS base, alanine, glycine, ammonia, dimethylamine, ethylamine, or any combination thereof.

5. The kit of any of claims 1-3, wherein the base is NaOH at a concentration of approximately 0.1 M.

6. The kit of any of claims 1-5, wherein the serum supplement is selected from the group consisting of fetal bovine serum, newborn bovine serum, adult bovine serum, fetal porcine serum, fetal equine serum, fetal carpine serum, fetal rabbit serum, fetal ovine serum, fetal chicken serum, or any combination thereof.

7. The kit of any of claims 1-5, wherein the aqueous base further comprises a cell growth medium.

8. The kit of any of claims 1-7, wherein the cell growth medium is a minimum essential medium (“MEM”).

9. The kit of any of claims 1-3 and 5-8, wherein the base is NaOH, the cell growth medium is 5X Minimal Essential Medium (“MEM”), the serum supplement is fetal bovine serum or bovine serum from newborn animals or adults, the organic chemical buffering agent is aqueous HEPES at a concentration of approximately 10-30 mM, and the collagen is atelopeptide-containing type I collagen at a concentration ranging from 1.0 to 4.4 mg/mL in 0.006 to 0.020 M aqueous HCl.

10. The kit of any of claims 1-9, wherein each of the collagen, the cell growth medium, the serum supplement, the organic chemical buffering agent, and the aqueous base is provided in a separate, sealed container.

11. The kit of any of claims 1-10, wherein the components are provided in the following ratios:
6.0 to 10.0 parts of the collagen, in solution;
1.0 to 3.0 parts of the cell growth medium;
0.5 to 1.5 parts of the serum supplement;
0.1 to 0.4 parts of the organic buffering agent; and
0.2 to 0.8 parts of the aqueous base.

12. The kit of any of claims 1-11, further comprising a telopeptide-containing type I collagen.

13. The kit of claim 12, wherein the telopeptide-containing type I collagen is provided at a concentration of 2.0 to 4.0 mg/mL in 0.004 to 0.016 M aqueous HCl.

14. The kit of claim 12 or 13, wherein the components are provided in the following ratios:
6.0 to 10.0 parts collagen solution;
1.0 to 3.0 parts cell growth medium;
0.5 to 1.5 parts serum supplement;
0.1 to 0.4 parts organic buffering agent;
0.2 to 0.8 parts aqueous solution comprising the strong base; and
1.5 to 2.5 parts telopeptide-containing type I collagen, in solution.

15. The kit of any of claims 1-14, wherein the aqueous base is 0.1 M NaOH provided at 4 volume percent of the total volume of solutions in the kit.

16. The kit of any of claims 1-10 and 12 wherein the components are provided in the following amounts: 16 volume percent of the cell growth medium, 8 volume percent of the serum supplement, 2 volume percent of the organic buffering agent, 70 volume percent of the collagen and 4 volume percent of the aqueous base.

17. A method of preparing a collagen medium matrix comprising:
preparing a first mixture comprising a cell growth medium, a serum supplement, and an organic buffering agent;
preparing a second mixture comprising a collagen and an aqueous base;
testing the pH of the second mixture;
adjusting the pH of the second mixture to a range of 6.8 to 7.6 by adding additional aqueous base in increments of approximately 1.0 to 4.0 μL, and combining the first and second mixtures.

18. The method of claim 17, wherein the base is a strong base at a concentration ranging from 0.05 to 0.25 M, the cell growth medium is 5X MEM, the serum supplement is fetal bovine serum or bovine serum from newborn animals or adults, the organic chemical buffering agent is aqueous HEPES at a concentration of 10 to 30 mM, and the collagen is atelopeptide-containing type I collagen at a concentration ranging from 2.0 to 4.0 mg/mL in 0.004 to 0.020 M aqueous HCl.

19. The method of claim 17 or 18, wherein the pH of the second mixture is adjusted by adding aqueous base in 2.5 μL increments.

20. The method of any of claims 17-19, wherein the aqueous base further comprises a cell growth medium.

21. The method of any of claims 17-20, wherein:
the first mixture comprises 130 to 200 parts of the MEM, 50 to 110 parts of the serum supplement, and 10 to 30 parts of the organic buffering agent;
the second mixture comprises, 60 to 84 parts of the aqueous base and 650 to 750 parts of the collagen at a concentration ranging from 2.0 to 4.0 mg/mL in 0.004 to 0.016 M aqueous HCl; and
wherein the aqueous base is a strong base at a concentration ranging from 0.05 to 0.25 M.

22. The method of any of claims 17-21, wherein the second mixture further comprises a telopeptide-containing type I collagen.

23. The method of claim 22, wherein the collagen is provided at a concentration ranging from 2.0 to 4.0 mg/mL in 0.004 to 0.016 M aqueous HCl, and the telopeptide-containing type I collagen is provided at a concentration ranging from 2.0 to 4.0 mg/mL in 0.004 to 0.016 M aqueous HCl.

24. The method of claim 22, wherein;
the first mixture comprises 130 to 200 parts of the MEM, 70 to 90 parts of the serum supplement, and 10 to 30 parts of the organic buffering agent; the second mixture comprises, 60 to 84 parts of the aqueous base, 600 to 660 parts of the collagen, at a concentration ranging from 2.0 to 4.0 mg/mL in 0.004 to 0.016 M aqueous HCl, and 40 to 100 parts of the telopeptide-containing type I collagen at a concentration ranging from 2.0 to 4.0 mg/mL in 0.004 to 0.016 M aqueous HCl; and
wherein the aqueous base is a strong base at a concentration ranging from 0.05 to 0.25 M.

25. A kit for preparing a collagen hydrogel medium matrix comprising the following components:
a collagen;
a cell growth medium;
an oncotically equivalent solution to serum;
an organic chemical buffering agent;
a cell growth medium; and
0.1 M NaOH.

26. The kit of claim 25, wherein the oncotically equivalent solution to serum is selected from the group consisting of bovine serum albumin, hydroxyethyl starch, dextran, and gelatin, or any combination thereof.

27. The kit of claim 25 or 26, wherein the cell growth medium is a minimum essential medium (“MEM”).

28. The kit of any of claims 25-27, wherein the cell growth medium is 5X Minimal Essential Medium (“MEM”), the oncotically equivalent solution to serum is bovine serum albumin, the organic chemical buffering agent is aqueous HEPES at a concentration of approximately 10-30 mM, and the collagen is atelopeptide-containing type I collagen at a concentration ranging from 1.0 to 4.4 mg/mL in 0.006 to 0.020 M aqueous HCl.

29. The kit of any of claims 25-28, wherein each of the collagen, the cell growth medium, the oncotically equivalent solution to serum, the organic chemical buffering agent, and the 0.1 M NaOH is provided in a separate, sealed container.

30. The kit of any of claims 25-29, wherein the components are provided in the following ratios:
6.0 to 10.0 parts of the collagen, in solution;
1.0 to 3.0 parts of the cell growth medium;
0.5 to 1.5 parts of the oncotically equivalent solution to serum;
1 to 0.4 parts of the organic buffering agent; and
0.2 to 0.8 parts of the 0.1M NaOH.

31. The kit of any of claims 25-30, further comprising a telopeptide-containing type I collagen.

32. The kit of claim 31, wherein the telopeptide-containing type I collagen is provided at a concentration of 2.0 to 4.0 mg/mL in 0.004 to 0.016 M aqueous HCl.

33. The kit of claim 31 or 32, wherein the components are provided in the following ratios:
6.0 to 10.0 parts collagen solution;
1.0 to 3.0 parts cell growth medium;
0.5 to 1.5 parts of the oncotically equivalent solution to serum;
0.1 to 0.4 parts organic buffering agent;
0.2 to 0.8 parts aqueous solution comprising the 0.1M NaOH; and
1.5 to 2.5 parts telopeptide-containing type I collagen, in solution.

34. The kit of any of claims 25-33 wherein the 0.1 M NaOH is provided at 4 volume percent of the total volume of solutions in the kit.

35. The kit of any of claims 25-29, 31-32, and 34, wherein the components are provided in the following amounts: 16 volume percent of the cell growth medium, 8 volume percent of the of the oncotically equivalent solution to serum, 2 volume percent of the organic buffering agent, 70 volume percent of the collagen and 4 volume percent of the aqueous base.
